(19) United States
(12) Patent Application Publication
(10) Pub. No.: US 2023/0101427 A1
(43) Pub. Date: Mar. 30, 2023

(54) FLOATING SYSTEM FOR PRODUCING MICROALGAE IN THE FORM OF BIOFILM

(71) Applicants: INALVE, Nice (FR); INSTITUT NATIONAL DE RECHERCHE EN INFORMATIQUE ET EN AUTOMATIQUE, Le Chesnay-Rocquencourt (FR)

(72) Inventors: Axel WAKEFIELD, Nice (FR); Freddy GUIHENEUF, Nice (FR); Olivier BERNARD, Carros (FR); Hubert BONNEFOND, Chateauneuf Villevieille (FR)

(21) Appl. No.: 17/910,150

(22) PCT Filed: Mar. 9, 2021
(86) PCT No.: PCT/EP2021/055902

§ 371 Date: Sep. 8, 2022

(30) Foreign Application Priority Data
Mar. 9, 2020 (FR) 2002312

(51) Int. Cl. C12M 1/00; A01G 33/00; C12M 1/09

(52) U.S. Cl. CPC C12M 21/02; A01G 33/00; C12M 23/56

(57) ABSTRACT

A system for producing microalgae in the form of biofilm, including a frame, a motor, at least one support and a web designed to receive the biofilm and in which the frame is configured to support the motor, the motor is configured to impart a rotational movement on at least one support, the at least one support is configured to have a rotational movement about an axis of rotation and to support and impart the rotational movement on the web, and the web is configured to at least partially surround the at least one support, wherein that the support is configured to have a non-negative buoyancy in a liquid.

### TECHNICAL FIELD OF THE INVENTION

The present invention relates to the field of cultivating microalgae. It has for particularly advantageous application the field of cultivating microalgae in the form of biofilm.

### PRIOR ART

Several types of microalgae exist and their cultures are different. For example there are planktonic microalgae and microalgae organised as biofilm. The objective of the present invention is to carry out a system allowing for the cultivating of microalgae in the form of biofilm exclusively. Indeed, the methods for collecting and processing microalgae cultivated as biofilm are entirely different and incompatible with the techniques for cultivating microalgae in planktonic form (i.e., free in the water). In fact, the production techniques for planktonic microalgae are cultivated in suspension in a liquid medium which is incompatible with a culture in the form of biofilm.

One of the techniques for cultivating microalgae in the form of biofilm is disclosed in document WO2015007724 A1. This document discloses a system including a frame, a pool of water, rollers for driving a biofilm and a motor making it possible to drive the rollers. The objective of this document is to improve the cultivating of microalgae in the form of biofilm by allowing for a regular exposure to a natural and/or artificial light of all the cells of the biofilm. To do this, the rollers are fastened on the frame which is itself fastened on the pool at variable immersion depths. The biofilm being driven by the different rollers from the immersed portion of the pool to an emerged portion.

The system disclosed in this document is very cumbersome to set up in that it requires a complex, voluminous and massive structure which is therefore expensive and energy-intensive.

One object of the present invention is therefore to propose a system that allows for the cultivating of microalgae in the form of biofilm that can be implemented more easily.

The other objects, characteristics and advantages of the present invention shall appear when examining the following description and accompany drawings. It is understood that other advantages can be incorporated.

### SUMMARY OF THE INVENTION

To reach this objective, according to an embodiment an assembly for producing microalgae in the form of biofilm is provided comprising at least one support and a web designed to receive the biofilm and wherein the at least one support is configured to have a rotational movement about an axis of rotation and to support and impart the rotational movement on the web, the web is configured to at least partially surround the at least one support, characterised in that the assembly has a non-negative buoyancy in a liquid. Preferably, the support and the web each have a non-negative buoyancy.

As the assembly is floating, the mass to be displaced for the rotation of the support is reduced limiting the costs. In addition, the assembly follows the level of the height of the liquid which also facilitates the following of the immersion of the microalgae over time. Even in the case of evaporation of the liquid, the support and the web will be in negative buoyancy and will make it possible to ensure a constant immersion of the microalgae.

According to another aspect, a system is provided for producing microalgae in the form of biofilm comprising a frame and an assembly such as described hereinabove wherein the frame is configured to receive the at least one support. Preferably, at least one from among the support and the frame is configured so that the system has a non-negative buoyancy in a liquid. According to another aspect, a system is provided for producing microalgae in the form of biofilm comprising a frame, at least one support and a web designed to receive the biofilm and wherein:
the frame is configured to receive the at least one support,
the at least one support is configured to have a rotational movement about an axis of rotation and to support and impart the rotational movement on the web;
the web is configured to at least partially surround the at least one support,

and wherein at least one from among the support and the frame is configured so that the system has a non-negative buoyancy in a liquid.

The system as a whole is floating in a liquid. The frame, the at least one support and the web receiving the biofilm together form an assembly having a non-negative buoyancy in a liquid. Having for example a support and/or a frame that floats, the system is lighter and more easily to assemble and disassemble. This reduction in mass comprises as a first advantage to limit the energy consumption for the setting into motion of the supports. In addition, the control of the temperature by evapotranspiration of the biofilm is made possible by the thermal inertia of the mass of the liquid on which the system floats. This allows for better control of the temperature by evapotranspiration of the biofilm and savings in energy consumption. Finally, the water becomes the bearing structure of the system, this reduces the needs of creating a substantial structure and allows for a lightening of the system as a whole as well as a simplification in the implementation thereof and a reduction in costs.

According to another aspect, the system is configured so that the support and the web form an assembly having a non-negative buoyancy. Preferably, the support and the web form an assembly that floats in a liquid, advantageously at least the support has a non-negative buoyancy, preferably, the support and the web respectively have a non-negative buoyancy.

### BRIEF DESCRIPTION OF THE FIGURES

The purposes and objects as well as the characteristics and advantages of the invention shall appear better in the detailed description of an embodiment of the latter which is shown by the following accompanying drawings wherein:

FIG. 1 shows a first embodiment of the system comprising a web stretched by several supports, and wherein the supports are horizontal and float in a liquid.

FIG. 2 shows an embodiment of the system similar to that of FIG. 1 but wherein said system is entirely immersed and floats as a suspension in a liquid.

FIG. 3 shows an embodiment of the system comprising several supports wherein the supports are horizontal and float on the surface in a liquid and wherein the web is distended.

FIG. 4 shows an embodiment comprising several supports wherein the supports are vertical and float in a liquid, and wherein each support supports its own web.

FIG. 5 shows an embodiment of a system comprising a motor supplied with energy by the movement of waves on the liquid.

FIG. 6 shows an embodiment comprising a single support floating on the surface of a liquid and wherein the frame supporting the motor is fastened on a solid surface. The imparting of a movement on the support is transmitted from the motor to the support in particular via a belt.

FIG. 7 shows an embodiment of the system wherein the motor is included in the support.

The drawings are given as examples and do not limit the invention. They form block diagrams intended to facilitate the comprehension of the invention and are not necessarily to the scale of the practical applications.

### DETAILED DESCRIPTION

Before beginning a detailed review of the embodiments of the invention, optional characteristics are mentioned hereinafter that can possibly be used in association or alternatively:
the support and the web form an assembly having a non-negative buoyancy,
the support and the web each have a non-negative buoyancy,
the system is configured to be at least partially immersed in the liquid;
the system is configured to be entirely immersed in the liquid and have a zero buoyancy;
the system comprises a displacement module configured to allow the system to alternatively have an entirely immersed position in the liquid and a partially immersed position;
the at least one support is substantially cylindrical and is preferably taken from among a roller or a tube;
the axis of rotation of the support is parallel to the longitudinal dimension of the support;
the system comprises a motor configured to impart the rotational movement about an axis of rotation of the support;
the displacement module comprises at least one ballast configured to vary and stabilise
the buoyancy of the system in the liquid.
the at least one ballast is arranged in the at least one support;
the axis of rotation of the support is mobile with respect to the frame,
the axis of rotation of the support is mobile with respect to the frame and follows the change in the height of water on which the support and/or the web and/or the frame are in non-negative buoyancy,
the axis of rotation of the support is mobile according to a movement comprising a non-zero vertical component with respect to the frame,
the system comprises a guide arm arranged between the frame and the support, preferably the guide arm is fastened to the frame by a point of fastening,
the guide arm is mobile about its point of fastening with the frame,
the guide arm is mobile about its point of fastening with the frame according to a movement comprising a non-zero vertical component with respect to the frame, preferably the guide arm is mobile in rotation about its point of fastening with the frame,
the system comprises several supports configured to be driven by at least one motor;
the system comprises several supports configured to be driven by several motors;
the system comprises several supports and at least one web and wherein each one of
the supports is configured to support and impart its movement on at least one web;
the system comprises several supports and a number of webs equal to the number of supports wherein each one of the supports is configured to support and impart its movement on a single web;
the system comprises a single support and a single web;
the motor is a power supply device configured to recover the energy of the movement of the waves on the surface of the liquid;
the at least one support is inflatable;
the axis of rotation of the at least one support is substantially horizontal;
the axis of rotation of the at least one support is substantially vertical.

The term “buoyancy” means the vertical thrust, directed upwards, that a fluid exerts on an immersed volume. Buoyancy always acts in the direction opposite gravity.

The buoyancy can be zero, i.e. the gravity and the pressure on the object are equal to the Archimedean thrust. In this case, the object is in suspension in the fluid.

The buoyancy can be negative, i.e. the gravity and the pressure exert a force that is greater than the Archimedean thrust. In this case, the object sinks into the fluid.

Finally, the buoyancy can be positive, i.e. the gravity and the pressure exert a force that is less than the Archimedean thrust. In this case, the object rises in the fluid.

It is specified that in the framework of the present invention, the verb “to float” means from its definition in that it allows an object to be borne by a liquid, and that it can be maintained therein at the surface or in suspension “drifting in midwater” without sinking therein. That is to say that the object has a non-negative buoyancy in this liquid.

It is known that the buoyancy is in particular a function of the density of the object with respect to the density of the fluid wherein it is totally or partially immersed.

Finally, it is specified that the term “motor” means a member transforming into mechanical energy an energy of a different nature. For example, the motor can be powered by a kinetic energy such as the movement of waves or the wind, by an electrical energy, thermal energy or any other type of energy. The motor can for example take the form of a mill, a turbine, or any other form making it possible to transmit a mechanical movement with an energy as input.

The system 1 for producing microalgae in the form of biofilm according to the present invention comprises at least one frame 100, advantageously a motor 200, at least one support 300 advantageously configured to float in a liquid 2 and support a web 400, the web 400 is configured to support microalgae in the form of biofilm. The motor 200 is configured to impart a movement on the support 300 and consequently on the web 400.

Advantageously, the system 1 allows for the cultivating of any type of microalgae, and preferably at least one of the varieties from among the following: Tisochisis lutea, Chlorella vulgaris, Navicula sp, Tetraselmis sp, Phaeodactylum tricornutum.

The system 1 can also comprise at least one spray device 500.

The system 1 is exposed to a light source 3. The web 400 comprises several portions. Thus, certain portions of the web 400 are in a direct exposure zone 3a allowing them to be exposed directly to the light source 3 while other portions are in an indirect exposure zone 3b that does not allow them to be directly exposed to the light source 3. The portions of the web 400 in the indirect exposure zone 3b benefit from reduced and even inexistant light.

The system 1 is configured so that the movement of the support 300 allows the different portions of the web 400 to pass from the direct exposure zone 3a to the indirect exposure zone 3b and vice versa.

When the system 1 is partially immersed, the system 1 is preferably configured so that the portions of the web 400 are successively in an immersed position and in an emerged position. This alternating in immersed/emerged position is carried out at a more or less regular frequency and preferably regular. This is in particular shown in FIGS. 1, 3, 5 and 6. In these embodiments, the system 1 can have between 0% and 99% of its surface emerged and preferably close to 95% of its surface emerged and preferably close to 99% of its surface emerged.

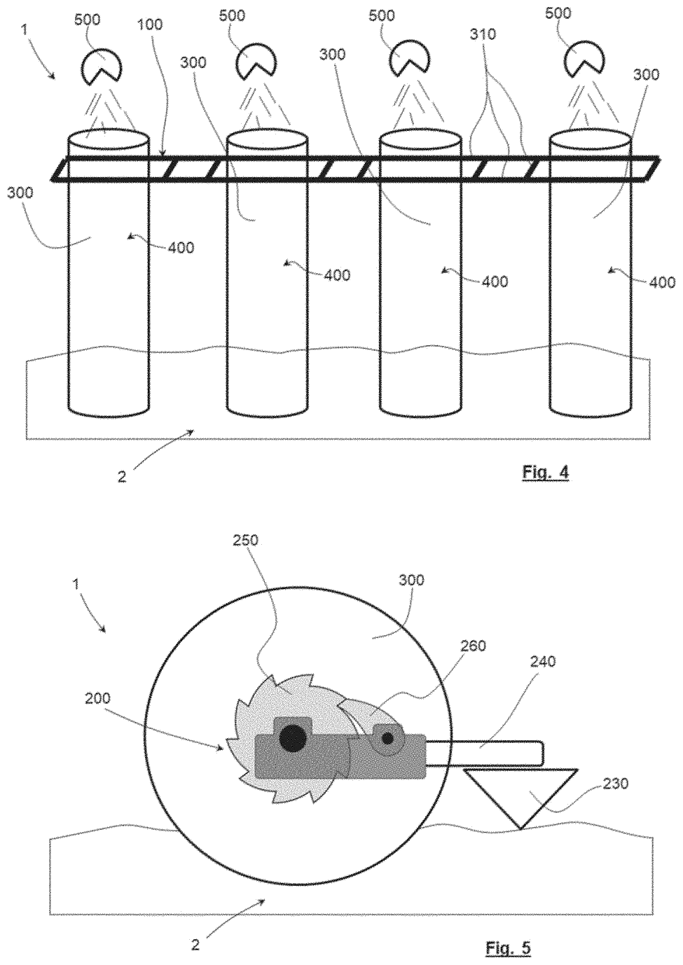

In certain embodiments, the immersion is total such as shown in FIG. 2. In other embodiments, such as shown in FIG. 4, portions of the support 300 are always immersed or emerged, and spray devices 500 humidify the emerged portions. These different embodiments and their designs are detailed hereinbelow.

The Frame 100

Advantageously, the frame 100 is configured to support at least the motor 200. The motor 200 is advantageously securely fastened to the frame 100 in such a way as to ensure the operation of the motor.

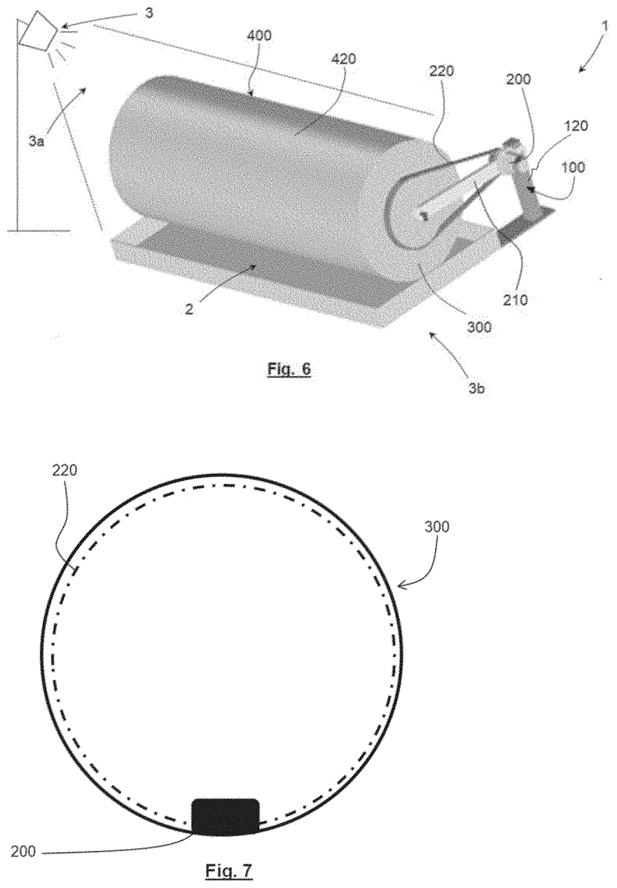

The frame 100 can be fastened on a rigid surface, such as shown in FIG. 6 for example, or totally supported by at least one support 300, such as shown in FIGS. 1 to 4 for example, or be supported in part by at least one support 300 and has a rigid surface, not shown in the figures. According to a possibility, the frame 100 is configured so that the system has a non-negative buoyancy and is therefore floating. According to a possibility, the frame 100 can have a non-negative buoyancy, i.e. the frame 100 is in itself floating. The frame 100 can advantageously comprise a hollow form filled or not with a gas or with a low-density foam. This characteristic makes it possible to increase the buoyancy of the system.

According to another possibility, the frame 100 has a negative buoyancy in itself. The system floats by the presence of at least one support 300 having a non-negative buoyancy, preferably of at least one support 300 and of a web 400, and possibly at least one float 320.

In certain embodiments, such as shown for example in FIGS. 1 to 4, the frame 100 connects in addition the supports together.

In the embodiments shown in FIGS. 1 to 4 the frame 100 comprises stringers 110. The stringers 110 make it possible to maintain between them in particular a plurality of supports 300 and/or at least one ballast 310 or at least one float 320. When the system comprises several supports 300, the frame 100 is advantageously configured to maintain the spacing between the different supports 300 for example at a substantially regular and preferably fixed distance. The maintaining at a regular and preferably fixed distance from the supports 300 is preferably carried out by the stringers 110. By way of example, the supports 300 and/or at least one ballast and/or float are fastened on the stringers 110. Preferably, a stringer 110 connecting several supports and/or ballasts and/or floats is configured to allow for a mobility comprising a non-zero component along a vertical axis between the connected elements. Thus, the system follows the movement and/or the level of the liquid 2, the system and more preferably the support 300 floats. The support 300 is maintained in contact with the liquid 2. The frame has in particular a flexibility authorising substantially vertical displacements of the connected elements of which in particular the support 300.

According to an advantageous possibility, the frame 100 is configured so that the support or supports 300 that have a non-negative buoyancy, float in the liquid 2. The frame 100 is advantageously configured so that the position of the at least one support follows the level of the liquid 2 wherein the system and preferably the support 300 floats. Preferably, the frame 100, more preferably the fastening between the motor 200 and the frame 100 constitutes a fixed reference standard with respect to the support 300. The support 300 has a mobility with respect to the frame 100, more precisely with respect to the fastening between the motor 200 and the frame 100, comprising a non-zero component along a vertical axis.

In FIG. 4 in particular, the frame 100 comprises at least one compartment making it possible to surround the support 300.

The frame 100 can comprise, in the embodiments where a single web 400 covers several supports 300, as shown for example in FIGS. 1 to 3, devices making it possible to guide the web 400 (not shown in the figures). These devices make it possible for example in FIGS. 1 and 2 to bring the web 400 closer to the frame 100. This makes it possible for example to more easily control the total volume of the system.

In FIG. 5, the frame 100 comprises the transmission arm 240 and at least one float 230.

In FIG. 6, the frame 100 comprises an upright 120, on which the motor 200 is advantageously mounted. The motor 200 is fastened on the upright 120. The system comprises a guide arm 210 providing the connection between the frame 100, more precisely the upright 120, and the support 200. The frame 100 is for example fastened on a solid surface. The solid surface can be a ground such as the edge or the bottom of a pool or the bank or the bottom of a watercourse or the coast or the bottom of a sea. The solid surface can also be a floating solid surface such as a pontoon, a boat, a barge, etc.

The Support 300

The system comprises according to a first possibility a support 300. According to a second possibility, the system comprises several supports 300. The rest of the description refers to a support without being limiting and can apply to all the supports.

The support 300 is advantageously configured to float in a liquid 2. This means that the support 300 floats on the surface or drifts in midwater. The term “floats on the surface” means when at least a portion of the support 300 is emerged.

The support 300 is advantageously of cylindrical shape and/or of axial symmetry and/or substantially conical. The support comprises an external surface. The external surface extends along a longitudinal dimension. The term “longitudinal dimension of the support” 300 means its largest dimension or also called its longitudinal extension axis. The support comprises two lateral surfaces corresponding to its bases at the ends of the external surface. The two lateral surfaces and the external surface define a interior volume. Preferably, the lateral surfaces are of the same dimension and of the same shape.

The support 300 is advantageously a roller.

The support 300 is advantageously an inflatable tube.

The support 300 is configured to have a rotational movement about an axis of rotation.

The movement of the support 300 is preferably a rotation about an axis of rotation. Advantageously, the axis of rotation of the support 300 is arranged parallel to the longitudinal dimension of the support 300. Preferably, the axis of rotation passes through the centre of each one of the lateral surfaces.

The external surface is configured to receive the web 400. The external surface is intended to be at least partially covered with a web 400. The external surface can comprise means of adherence and/or of fastening the web 400. Through a non-limiting example, the external surface can comprise successions of cavities and asperities making it possible to increase the adherence with the web 400. Means of fastening can for example and in a non-limiting manner be a surface with bridges configured to cooperate with hooks carried by the web 400. The elements of fastening and of adherence can be diverse and varied and are not limited to the examples hereinabove. The support 300 is configured to impart its movement on at least one web 400.

In an embodiment of the invention, the support 300 comprises a length comprised between 2 and 500 metres and preferably between 2 and 200, preferably between 2 and 100 metres, more precisely between 5 and 200 metres, more precisely between 5 and 50 metres. By way of example, the support 300 has a diameter comprised between 0.10 and 3 metres and preferably between 0.5 metre and 1.5 metres.

Advantageously, the support 300 is made of an impermeable material such as polyvinyl chloride for example.

Preferably, the support 300 is made of an inflatable flexible material such as a fabric for example. In this embodiment, the pressure inside the support 300 is greater than 10 millibars (mBar), and preferably greater than 30 mBar. This advantageous construction makes it possible to improve the reduction in mass of the support 300. Thus, the energy required for setting the support 300 in motion is greatly reduced. In addition, the inflatable support 300 allows for an assembly, a disassembly, a transport and a storage of the entire system 1 that is facilitated.

According to a possibility, the support 300 is permeable to gas, in particular to carbon dioxide (CO₂) in order to allow for the diffusion of the CO₂ of the support to the algae biofilm. According to another possibility that is compatible with the preceding one, the support 300 is permeable to a concentrated nutrient medium. In this possibility, the nutrient medium is stored in the support 300. In an embodiment, the interior volume of the support 300 is full. In this embodiment, the interior volume of the support 300 is filled with a material allowing it to float such as for example a polyester or polystyrene foam. It is possible to vary the buoyancy of the support 300 by varying the density of the foam. Indeed, the lower the density of the foam is, the higher the buoyancy of the support 300 will be. On the other hand, with a foam having a higher density, the buoyancy of the support 300 will be lessened.

In another embodiment, the interior volume of the support 300 is hollow and filled with gas preferably under pressure (pressure greater than 10 mbar). In this embodiment, the buoyancy of the support 300 is variable according to the quantity of gas under pressure injected into the interior volume of the support 300. The embodiment has the advantage being easily implemented and transportable. The support 300 is advantageously inflatable. The support 300 advantageously has a rigidity that is sufficient to ensure a transmission of the movement of rotation over its entire longitudinal dimension. The support 300 preferably has a hardness allowing the scraping of the algae biofilm present on the surface of the web.

In this embodiment, the support 300 can comprise devices configured to operate as a ballast. For example, the support 300 can comprise at least one valve and/or at least one air pump and/or a water pump making it possible to vary the volume of gas inside the support 300. This variation can for example allow the system 1 to vary its buoyancy between a positive buoyancy, i.e. the support is partially immersed, for example with less than 5 centimetres of the support 300 immersed and a zero buoyancy, i.e. the support 300 is in balance in a liquid, i.e. it is according to the expression the support 300 is in suspension in a liquid “drifting in midwater”. The passage from a partially emerged position to a completely immersed position can be carried out by granting the system 1 with a negative buoyancy. Such a negative buoyancy is transient and solely has for objective the variation in the immersion of the system 1. In the case where the support 300 is in balance in a liquid, the entire support can be submerged or at any intermediate position. This variation can make it possible to adapt the conditions for producing microalgae according to their needs in air, light or liquid 2. Likewise, in case of installation in a natural environment such as for example at sea or in a lake, the immersion of the system 1 in case of difficult meteorological conditions such as violent winds or a substantial swell for example a total immersion can make it possible to prevent damage to the system.

In another embodiment of the invention, the support 300 has a fixed density and the system comprises at least one ballast 310 or at least one float 320 advantageously positioned on the frame 100.

According to an embodiment, the support 300 is arranged in such a way that its longitudinal dimension is vertical. The term “vertical” means parallel to the direction of gravity. According to another embodiment, the support 300 is arranged in such a way that its longitudinal dimension is horizontal. The term “horizontal” means the direction normal to the vertical.

The mass of the support 300 is advantageously comprised between 2 and 50 kilogrammes per m² of ground footprint and preferably between 5 and 20 kilogrammes per m² of ground footprint. In certain embodiments, the system comprises several supports 300 such as shown as examples in FIGS. 1 to 4.

In these embodiments, the supports 300 can be positioned substantially horizontally (FIGS. 1 to 3) or substantially vertically (FIG. 4). The supports 300 are preferably, but not in a limited manner, substantially parallel to one another.

In these embodiments, the supports 300 are maintained together via the frame 100. The maintaining is preferably carried out by at least one of the lateral faces of the supports 300 and can be carried out by the two faces.

In the embodiment shown in FIG. 4 and wherein the supports 300 are vertical, the supports 300 can comprise lests on and/or in their immersed portions. The lests are configured to stabilise the supports 300.

The Ballast 310 and the Float 320

A ballast is a device equipped with means making it possible to vary its buoyancy. Conventionally, a ballast can in its interior volume vary the pressure of a gas or the proportion of gas/liquid so as to modify its own buoyancy and the buoyancy of the system to which it is connected. In certain embodiments provided with at least one and preferably several ballasts 310, the latter can communicate with each other or not. Thus, each ballast 310 can communicate its buoyancy to the other ballasts 310, or to an external module for managing the buoyancy of the system 1. The communication can also be a fluidic communication making it possible to vary between them the levels of gas/liquid present in the ballasts 310. This makes it possible in particular to vary the buoyancy of only a portion of the system 1 so as for example to modify the attitude of the system. In other words, to vary the inclination of the system with respect to a plane comprising the surface of the liquid 2. A ballast 310 can be configured to have the role of both a float and that of a lest. In its lest function, the ballast 310 allows for better stability of the floating system 1.

A float 320 is a floating device that makes it possible to improve the buoyancy of the system 1. Its buoyancy is fixed. This is the case for example of a buoy. To do this, the float 320 is either filled with a gas, or with a low-density foam. Advantageously, the float 320 is fastened on the frame 100.

The Web 400

The web 400 is configured to cover at least partially the support 300 and more preferably the external surface of the support 300. The support 300 and the web can be a single and same element. The web 400 is configured to allow for the fastening of microalgae in the form of biofilm. To this effect, the web 400 is either configured to directly receive the algae biofilm or the web 400 comprises a coating that can be formed by an additional web at least partially covering the web 400. The additional web is however configured to receive the algae biofilm. The web 400 is at least partially under the surface of the liquid 2. The web 400 comprises several portions of which at least certain portions are immersed. There can also be emerged portions. The immersed and emerged portions vary according to time and the movement of the web 400.

According to a possibility, the web 400 has a non-negative buoyancy, i.e. the web floats. More preferably, the support 300 and web 400 assembly has a non-negative buoyancy.

Advantageously, microalgae have a thermal preferendum, i.e. the temperature of the medium wherein they are cultivated has a direct influence with the speed of their growth.

Thus, if the temperature is too low or too high with respect to their thermal preferendum, the growth of the microalgae slows down and the population even decreases.

In fact, it is preferred to maintain a stable temperature during the growth of microalgae.

Note that two mechanisms contribute to maintaining the temperature in a thermal preferendum compatible with growth, and in any case by preventing fatal temperatures: evapotranspiration and thermal inertia of the mass of water wherein the system 1 floats. Advantageously, alternating the immersion and the emersion of the web 400 allows for a better control of the thermal preferendum of the habitat of the microalgae.

In certain embodiments, the web is entirely plunged in the liquid 2 but retains a neutral or negative buoyancy.

Preferably, the two ends of the web 400 are connected together so as to form a loop. In another non-preferred embodiment of the invention, each one of the ends of the web 400 is fastened on the external surface of a support 300 and is therefore immobile with respect to the support. The web 400 comprises a first face 410 and a second face 420. The first and second faces 410, 420 can be carried out by a single layer or by several layers superimposed on each other.

The first face 410 is facing at least one support 300. The first face 410 is configured to allow for the contact and the adherence with said support 300. The first face 410 is preferably carried out with a material allowing for the adhesion to the support 300 such as for example a fabric, a canvas, a polyester or polyurethane web, etc. The adhesion can, for example, be carried out by friction, or with asperity/cavity alternatives in the first face 410. However, any other material allowing for the carrying out of a web 400 can be used for this face.

The adhesion of the first face 410 to the support 300 allows said support 300 to impart its movement on the web 400. Thus the web 400 is mobile at least with respect to the frame 100. In certain embodiments such as shown in FIGS. 1 to 3, the web 400 is also mobile with respect to the support 300.

In these embodiments, the first face 410 is configured to sufficiently adhere with the support 300 so as to allow for the transmission of the movement of the support 300 to the web 400 while still allowing for a mobility of the web 400 regarding said support 300. In other embodiments such as shown for example in FIGS. 4 and 6, the first face 410 is relatively fixed to the support 300. In these embodiments, the first face 410 then comprises elements for fasting to the support 300. The elements for fastening can for example be a glue, a surface in form of a bridge cooperating with a surface comprising hooks on the support 300, staples, nails, screws or any other element allowing for a fastening between the web 400 and the support 300. Advantageously the fastening is reversible in order to be able to separate the web 400 and the support 300.

The second face 420 is configured to be on the side opposite the first face. The second face 420 is either configured to allow for the fastening of the microalgae in the form of biofilm or to receive a preformed covering of an additional web able to receive the algae. The second face 420 advantageously does not have contact with the support 300. Advantageously, the material used for the fastening of the microalgae in the form of biofilm is preferably rough having cavities or microcavities. For example, the second face can be made of one of the following materials: cotton, jute cloth, polyethylene, polyurethane, biopolymer or of polyester.

In an embodiment the first and second faces 410; 420 of the web 400 are spaced apart from one another so as to form an internal volume. In this embodiment, a gas is injected into the internal volume so as to inflate the web 400. According to a possibility compatible with the preceding one, a nutrient medium is injected into the internal volume. The web 400, and more particularly the second face 420 is then porous to the nutrient medium so as to feed the microalgae. This embodiment makes it possible to improve the buoyancy of the system 1 by adding a volume of gas to the system. In this embodiment, the web 400 comprises at least one device, such as for example a valve and/or an air pump, making it possible to vary the quantity of gas in the interior volume so as to be able to vary the buoyancy.

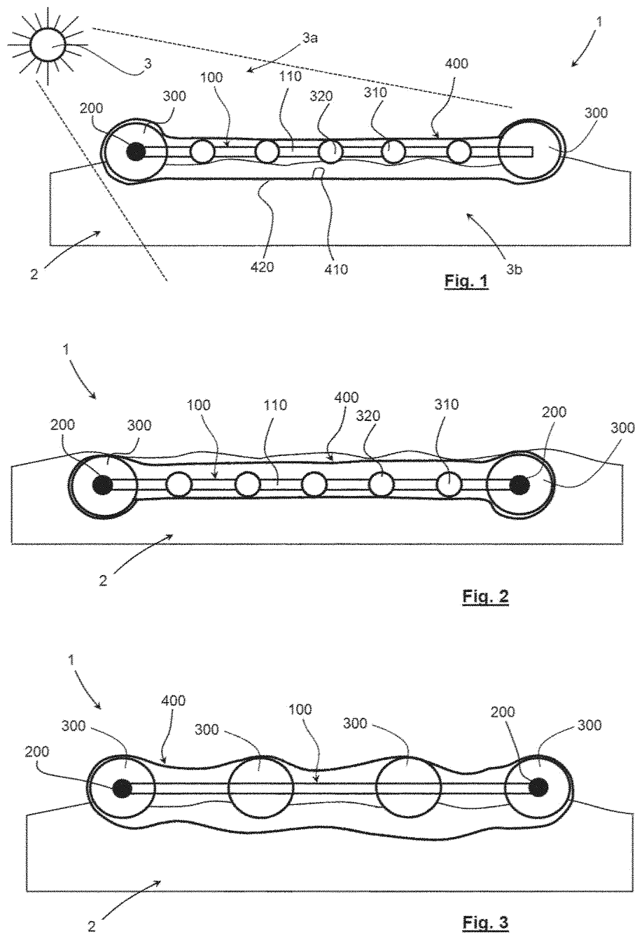

In an embodiment comprising several supports 300, a single web 400 can surround several supports 300 in such a way as to form a conveyor. In this embodiment, the web 400 can be stretched at least partially between the supports 300, such as shown in FIGS. and 1 and 2, or be distended between the supports 300 such as in FIG. 3. In this embodiment particularly, the system 1 comprises tensioning elements of the web 400. The tensioning elements of the web 400 are configured to make it possible to stretch the web 400 so as to facilitate the scraping of the microalgae. An advantage of this solution is to increase the culture surface for a given system 1. In another embodiment, such as shown for example in FIGS. 4 and 6, each support 300, when there are several of them, is completely surrounded by a single web 400. This advantageous solution makes it possible for example to vary the characteristics of the webs 400 according to the type of production on each one of said webs 400. The web 400 is configured to allow for the scraping of the microalgae. Thus, even in the case where the web is distended, the tensioning elements make it possible to ensure the scraping.

The Motor 200

The motor 200 being however configured to impart on the at least one support 300 a movement preferably of rotation with respect to the frame 100. Advantageously, the frame 100 receives the motor 200. The frame 100, advantageously receiving the motor 200, forms a reference standard with respect to the movement of the support.

The motor is configured to have at the output a preferred movement of rotation.

The system 1 can comprise a single motor 200 or a plurality of motors 200 for one or more supports 300.

For example such as shown in FIG. 1, the system 1 comprises a single motor for several supports 300. In FIG. 2 for example the system 1 comprises several motors 200 for several supports 300. In FIG. 4, the system 1 comprises one motor per support 300. Finally, in FIG. 6, a single motor 200 is present on a single support 300. Other combinations are of course possible, for example and in a non-limiting manner, having several motors 200 per support 300, in particular connected to each one of the lateral surfaces of the support 300.

According to an embodiment, the system comprises a connection device between the motor 200 and at least one support 300. The connection device is configured to ensure the transmission of the movement of the motor 200 to the support 300 and more preferably impart a movement of rotation to the support 300. According to a possibility, the motor 200 is connected to the external surface of at least one support 300. According to a possibility, the motor 200 is connected to at least one of the lateral surfaces of the support 300. The connection device 220 can for example be a belt, a cable, a chain, a rack or a toothed wheel or any other connection element. The connection can for example be provided via a disc positioned as a protuberance of said lateral surface or of a toothed wheel (not shown in the figures).

FIG. 6 shows an embodiment of the connection between the motor 200 and the support 300. In this embodiment, a guide arm 210 maintains in position the support 300 at its axis of rotation at a predefined distance from the motor 200. A connection device 220 provides a connection between the motor 200 and a lateral surface of the support 300. The connection device 220 can be for example here a belt. The connection can for example be provided via a disc positioned as a protuberance of said lateral surface or of a toothed wheel (not shown in the figure). The connection device 220 allows for the transmission of the movement of rotation of the motor 200 to the support 300. The connection device 220 can be replaced with a chain for example. In this embodiment, the guide arm 210 does not support the support 300 and has only a guiding function. Said support 300 is supported by the liquid thanks to its buoyancy. The guide arm 210 only has a guide function 210 allowing for the setting up of the connection device 220 and the transmission of the movement. The guide arm 210 has a mobility with respect to the frame 100 and here to the upright 120. The guide arm 210 connects the frame 100 to the support 300. The guide arm 210 is mobile according to a movement having a non-zero vertical component with respect to the frame 100 and more particularly to the upright 120. The mobility of the guide arm is done at its point of fastening on the frame 100, more precisely on the upright 120.

The support 300 is advantageously mobile with respect to the frame 100.

According to an embodiment, the position of the support 300 with respect to the frame 100 is variable according to the buoyancy of the support 300. More precisely, it is the position of the axis of rotation of the support 300 with respect to the frame 100 that is variable according to the buoyancy of the support 300. The position of the support 300 with respect to the frame 100 is variable according to the level of the liquid 2 wherein the system and preferably the support 300 floats. This arrangement allows the support to follow the level of the liquid 2 and in particular in the case of a pool of liquid 2, the level of the liquid can vary due to the evaporation of the latter, the level of buoyancy of the support therefore remains identical without requiring a complex control. Preferably, the support 300 has a mobility with respect to the frame 100 comprising a non-zero component according to a vertical axis. The vertical axis being perpendicular to the surface of the liquid 2.

According to a possibility, the connection device is configured to allow for a mobility of the support 300 with respect to the frame 100 comprising a non-zero component along a vertical axis. This configuration allows the system to ensure that the support 300 floats in the liquid and thus follows the variations in the level of the liquid.

Other embodiments are of course possible. For example, the output of the motor 200 can be directly connected to a lateral surface of the support 300 as shown for example in FIGS. 1 to 5.

In these embodiments, the connection between the motor 200 and the support 300 can for example se faire through pinions, but via rollers bearing on the support 300 or any other means that making it possible to transmit the movement of the motor 200 on the support 300. For example in an embodiment, the frame 100 can be mounted free in rotation inside the support 300. In this case, the motor 200, supported by the frame 100, can be configured to have an output connected to the internal face of the support 300 and thus drive a rotation of the support 300 with respect to the frame 100, thanks to the connection device 220. This solution makes it possible to have a system with a minimalized size. An example of this embodiment can be seen in FIG. 7. In this embodiment, the motor 200 is associated with a lest and is enclosed inside the support 300. Preferably the support 300 is rigid, either thanks to the composition of its materials or thanks to its internal pressure. In addition, the support 300 is preferably tight and comprises, along its internal wall a connection device 220 taking the form of a rack and/or of a cable. The motor 200 being configured to cooperate with the rack and/or the cable and drive the support 300 in rotation. The weight of the motor is configured to rotate the structure. A connection with a sealed rotating connector makes it possible to bring electricity to the motor 200 inside the support 300.

Preferably, the motor 200 is an electric motor comprising a stator and a rotor. The stator is preferably fastened on the frame 100. The rotor directly or indirectly connected to the support 300 allows for the rotation of said support 300 with respect to the frame 100. The motor 200 can also be solely mechanical such as shown in FIG. 5. The storage and the restitution of energy being done for example through at least one spring configured to release its energy over time.

The supply of electrical energy to the motor can be diverse and varied. For example, the motor can be connected to batteries (lithium-ion, lead, etc.), or to any electrical grid. Moreover, the supply of energy directly in the motor 200 or to a battery can also be done through renewable energy for example solar energy, wind turbine, by the movement of waves or tides.

FIG. 5 shows an embodiment of the supply in energy by a solution of recovering the energy generated by waves. In this embodiment, a float 230 is connected to a transmission arm 240 with a toothed wheel 250. The transmission arm 240 preferably has a length greater than the radius of the lateral surface of the support 300. The transmission arm bears a ratchet 260. The ratchet 260 is configured to allow for a rotation of the toothed wheel 250 according to a first direction of rotation, and to prohibit the rotation of the toothed wheel 250 in a second direction of rotation opposite the first direction of rotation.

The toothed wheel 250 can be connected to the support 300 either directly to the frame 100, or through a device for recovering energy so as to supply the motor with energy. In this embodiment, the float 230 follows the movement of the waves of the liquid 2 with a slight offset with respect to the support 300. This difference in movement is transmitted by the transmission arm 240 to the toothed wheel 250 by the leverage effect and makes it possible to actuate the rotation of the toothed wheel 250. According to the length of the transmission arm 240, it is possible to vary the difference in movement of the float 230 with respect to the support 300. This difference makes it possible to set the toothed wheel 250 in motion.

Thus, the longer the transmission arm 240 is, the smaller the difference in movement required between the float 230 and the support 300 to set the toothed wheel 250 in motion is. In other words, a low swell will make it possible to set the toothed wheel 250 in motion.

This embodiment uses the principles of recharging automatic watches comprising a mechanical motor.

In the case where the toothed wheel is carried by the frame and directly transmits its movement of rotation to the support 300, then the assembly of the system for recovering the energy of waves can be considered as the motor 200.

Finally, it is possible to couple the supplies of the motor 200. For example, a switch can allow the motor 200 to be supplied either by a renewable source of energy, or by a battery so as to be able to adapt to the different meteorological conditions. The extra energy produced by the renewable source of energy can be used to charge the batteries.

Spray Device 500

The system 1 can comprise at least one and preferably several spray devices of a liquid. For example, in the embodiment shown in FIG. 4, spray devices, preferably at least one per support 300, making it possible to guarantee the correct supply with liquid in particular with a nutritious mixture and therefore the correct humidification the second face 420 of the web 400. This supply with liquid and/or with nutritious elements makes it possible to better control the characteristics required for the production of microalgae in the form of biofilm.

The spray device can be fastened to the frame 100 or have its own structure.

The spray device 500 is provided with a pump that makes it possible to draw from a reserve the liquid that is emitted. The reserve can be the liquid 2 wherein the system 1 floats.

The Liquid 2

The liquid 2 is a liquid that allows for the cultivating of microalgae in the form of biofilm. Advantageously, the liquid 2 is water that can be salt or fresh water with or without an additive such as supplies with nutrients for example. The liquid 2 can be contained in a natural body of water such as a lake, a watercourse or the sea.

The liquid 2 can also be contained in an artificial pool, i.e. in a human construction for example a masonry tank or made of wood or of any other materials. The carrying out of an artificial pool allows in particular for a better control of the characteristics of the liquid 2 such as its potential of hydrogen (pH), its temperature, or its composition in nutritious elements and/or in waste or its level or the presence of bacteria in the liquid 2. The artificial pool is open in its upper portion and exposed at least partially to at least one light source 3.

The Light Source 3

The light source 3 can be a natural source such as the sun or an artificial light making possible in particular a supply in particular with ultraviolet light required for the cultivating of microalgae. During the use of a natural body of water, the supply with light is carried out mainly by the sun. However, the system can comprise an additional light source 3 in addition to the main light source 3 thus making it possible to reinforce the light or the supply with ultraviolet light when the climatic conditions are not optimal and/or to expose to the light portions different from those exposed to the main light source 3.

### CERTAIN SPECIFIC EMBODIMENTS

FIG. 1 shows an embodiment of the system 1. The system 1 floats in a liquid. The system 1 comprises a first and a second support 300 connected and positioned substantially horizontally and parallel to each other. The first and second supports 300 are connected together by the frame 100 via lateral surfaces of the two supports 300. Advantageously, the frame 100 and the first and second supports 300 are connected at the axis of rotation of the supports 300. Preferably, the support 300 is mobile in rotation about its axis of rotation. Preferably, the support 300 is mobile following a movement having a non-zero vertical component with respect to the frame. More precisely, the longitudinal axis of the support 300 preferably confounded with the axis of rotation of the support is mobile, advantageously following a movement having a non-zero vertical component, with respect to the frame 100, more precisely to the frame at its fastening to a float or to the second support. A motor 200 is arranged on the frame 100 for example at an end. The motor 200 is configured to drive in rotation the first support 300. The frame 100 comprises between the two supports 300 several floats 320 and/or ballast 310. A web 400 extends between the two supports 300 and drives them at least partially. The web 400 is driven in rotation around two supports 300 by the rotation of the first support 300 so that the portions in the direct exposure zone 3a alternate their positions with the portions in the indirect exposure zone 3b. The web 400 is relatively stretched between the first and second supports 300. The web 400 extends along a plane in contact with the external surface of the supports 300. The web 400 extends in a straight line between two external surfaces of two supports 300. In this embodiment portions of the web 400 are exposed to the open air and other portions of the web 400 are immersed in the liquid 2. FIG. 2 shows an embodiment identical to the one shown in FIG. 1 but wherein the entire system 1 has zero buoyancy. The system 1 floats as drifting in midwater. Advantageously the entire web 400 is immersed.

According to a possibility of this embodiment, a second motor 200 drives the second support 300 in rotation.

FIG. 3 shows an embodiment similar to the two first embodiments with the difference that the floating system 1 comprising four supports 300 and no float. The supports 300 integrate the function of a float and/or ballast. The web 400 comprises portions exposed to the open air and immersed portions. The web 400 is distended between the two most extreme supports 300. The web 400 is loose between two external surfaces of two supports 300.

FIG. 4 shows an embodiment with four supports 300 positioned substantially vertically and parallel to one another. The supports 300 comprise an immersed portion and an emerged portion respectively according to the buoyancy of the support 300. The supports are connected together by the frame 100. Spray devices 500 make it possible to humidify the non-immersed portions of the supports 300. A web 400 surrounds each support 300. In this embodiment, lests (not shown in the figure) can be positioned on the immersed portions so as to stabilise the system 1. Finally, FIG. 6 shows an embodiment wherein the system 1 comprises a single support 300 positioned substantially horizontally. The frame 100 is fastened to a solid surface and the support 300 floats. The support 300 is for example partially immersed (five centimetres). A single web 400 preferably completely surrounds the support 300. The motor 200 makes it possible to drive in rotation the support 300 so that the portions of the web 400 located in the open air and in the direct exposure zone 3a of the light alternate their positions with the portions of web 400 located in the immersed portion of the support 300 and/or in the indirect exposure zone 3b.

The invention is not limited to the embodiments described hereinabove and extends to all the embodiments covered by the claims.

1. An assembly for producing microalgae in the form of biofilm comprising at least one support and a web designed to receive the biofilm and wherein
the at least one support is configured to have a rotational movement about an axis of rotation and to support and impart the rotational movement on the web;
the web is configured to at least partially surround the at least one support, wherein the assembly has a non-negative buoyancy in a liquid.

2. The assembly according to claim 1 wherein the support and the web each have a non-negative buoyancy.

3. A system for producing microalgae in the form of biofilm comprising a frame, and an assembly according to claim 1 wherein the frame is configured to receive the at least one support.

4. The system according to claim 3 wherein at least one from among the support and the frame is configured so that the system has a non-negative buoyancy in a liquid.

5. The system according to claim 3 wherein the at least one support is cylindrical and is preferably taken from among a roller or a tube.

6. The system according to claim 3 comprising a motor configured to impart the rotational movement about an axis of rotation of the support.

7. The system according to claim 3 wherein the axis of rotation of the support is mobile with respect to the frame.

8. The system according to claim 3 wherein the axis of rotation of the support is mobile according to a movement comprising a non-zero vertical component with respect to the frame.

9. The system according to claim 3 comprising a guide arm arranged between the frame and the support.

10. The system according to claim 9 wherein the guide arm is mobile about its point of fastening with the frame.

11. The system according to claim 10 wherein the guide arm is mobile about its point of fastening with the frame according to a movement comprising a non-zero vertical component with respect to the frame.

12. (canceled)

13. (canceled)

14. The system according to claim 3 wherein the system comprises a displacement module configured to allow the system to alternatively have an entirely immersed position in the liquid and a partially immersed position.

15. The system according to claim 14 wherein the displacement module comprises at least one ballast configured to vary and stabilise the buoyancy of the system in the liquid.

16. The system according to claim 15 wherein the at least one ballast is arranged in the at least one support.

17. The system according to claim 3 comprising several supports configured to be driven by at least one motor.

18. The system according to claim 3 comprising several supports configured to be driven by several motors.

19. The system according to claim 3 comprising several supports and at least one web and wherein each one of the supports is configured to support and impart its movement on at least one web.

20. The system according to claim 3 comprising several supports and a number of webs equal to the number of supports wherein each one of the supports is configured to support and impart its movement on a single web.

21. The system according to claim 3 wherein the system comprises a single support and a single web.

22. The system according to claim 3 wherein the motor is a power supply device configured to recover the energy of the movement of the waves on the surface of the liquid.

23. The system according to claim 3 wherein the at least one support is inflatable.

24. The system according to claim 3 wherein the axis of rotation of the at least one support is horizontal.

25. The system according to claim 3 wherein the axis of rotation of the at least one support is vertical.
